(19) United States
(12) Patent Application Publication
(10) Pub. No.: US 2014/0322804 A1
(43) Pub. Date: Oct. 30, 2014

(54) PHOTOBIOREACTORS AND CULTURE BAGS FOR USE THEREWITH

(71) Applicants: Sabin Boily, Chambly (CA); Serge Bujold, Noyan (CA); Erwann Fraboulet, Saint-Anaclet-de-Lessard (CA)

(72) Inventors: Sabin Boily, Chambly (CA); Serge Bujold, Noyan (CA); Erwann Fraboulet, Saint-Anaclet-de-Lessard (CA)

(21) Appl. No.: 14/008,941

(22) PCT Filed: Apr. 2, 2012
(86) PCT No.: PCT/CA2012/000330

§ 371 Date: Jan. 29, 2014

Related U.S. Application Data
(60) Provisional application No. 61/470,002, filed on Mar. 31, 2011.

(51) Int. Cl. C12N 1/12

(52) U.S. Cl. CPC C12N 1/12
USPC 435/292.1

(57) ABSTRACT

There is provided a culture bag for use in a photobioreactor. The bag can comprise at least one wall having at least one inlet disposed at a first end portion of the at least one wall or adjacently thereto. The at least one wall defines an internal chamber for receiving a culture medium. The bag also comprises at least one injector for injecting a gas inside the bag, the at least one injector being disposed at a second end portion of the at least one wall or adjacently thereto. The bag also comprises at least one outlet for harvesting a content of the bag, the at least one outlet being disposed at the second end portion of the at least one wall or adjacently thereto. The bag can be translucent or transparent and be effective for holding and sealingly maintaining the culture medium inside the bag and inside the photobioreactor. There is also provided a photobioreactor and a photobioreator modular system.

### FIELD OF THE DISCLOSURE

The present disclosure relates to the field of photobioreactors that can be used, for example, for the production of microalgae. In particular, the present disclosure relates to photobioreactors and to culture bags that can be used with such devices.

### BACKGROUND OF THE DISCLOSURE

Several systems are known in the art for producing microalgae. However, several of them are either very costly to acquire and/or to operate. Moreover, several proposed technologies do not allow for producing, at low costs, high quality microalgae. Another problem encountered is the space required (surface area i.e. several square foot or square meters) by such systems. In fact, when using indoor systems, many of these systems require a lot of space (footprint), which can be a considerable drawback.

### SUMMARY OF THE DISCLOSURE

It would thus be highly desirable to be provided with an apparatus that would at least partially solve one of the problems previously mentioned or that would be an alternative to the existing technologies.

According to one aspect, there is provided a culture bag for use in a photobioreactor, the bag comprising:
a first wall having an inlet and a second wall;
an injector for injecting a gas inside the bag, the injector being disposed adjacently to the second wall;
optionally at least one inlet disposed on a first side wall of the bag, the at least one inlet being effective for receiving further elements such as a sensor or a sampling probe; and
an outlet for harvesting a content of the bag,
   the bag being translucent or transparent and being effective for holding and sealingly maintaining a culture medium.

According to another aspect, there is provided a culture bag for use in a photobioreactor, the bag comprising:
a first wall having an inlet;
a second wall;
at least one side wall disposed between the first and the second walls and connected thereto;
an injector for injecting a gas inside the bag, the injector being disposed adjacently to the second wall;
optionally at least one port disposed on a first side wall of the bag, the at least one port being effective for receiving at least one element chosen from a sensor, a sampling loop and a probe; and
an outlet for harvesting a content of the bag,
the bag being translucent or transparent and being effective for holding and sealingly maintaining a culture medium.

According to another aspect, there is provided culture bag for use in a photobioreactor, the bag comprising:
at least one wall having at least one inlet disposed at a first end portion of the at least one wall or adjacently thereto, the at least one wall defining an internal chamber for receiving a culture medium;
at least one injector for injecting a gas inside the bag, the at least one injector being disposed at a second end portion of the at least one wall or adjacently thereto;
optionally at least one port disposed on the at least one wall of the bag, the at least one port being effective for receiving at least one element chosen from a sensor, a sampling loop and a probe; and
at least one outlet for harvesting a content of the bag, the at least one outlet being disposed at the second end portion of the at least one wall or adjacently thereto,
the bag being translucent or transparent and being effective for holding and sealingly maintaining the culture medium inside the bag and inside the photobioreactor.

According to another aspect, there is provided a photobioreactor comprising:
a culture bag dimensioned for receiving a culture medium;
a housing defining an internal chamber dimensioned for receiving the culture bag, the housing comprising at least one wall having at least one translucent or transparent portion comprising a translucent or transparent material and optionally at least one opaque portion comprising at least one opaque material; and
at least one lighting element disposed adjacently to the translucent or transparent portion so as to provide light inside the chamber.

According to another aspect, there is provided a photobioreator modular system comprising a plurality of photobioreactor units wherein at least one of the units is a photobioreactor as defined in the present disclosure.

### BRIEF DESCRIPTION OF THE DRAWINGS

The following examples are presented in a non-limitative manner.

FIGS. 1A and 1B represent front elevation views of two different examples of culture bags as described in the present disclosure;

FIG. 2 is a schematic representation of examples of a culture bag, a photobioreactor, and photobioreactor systems as described in the present disclosure;

FIG. 3 is a top view an example of a photobioreactor as described in the present disclosure in which a culture bag has been removed;

FIG. 4 is a top view of another example of a photobioreactor as described in the present disclosure in which a culture bag has been inserted;

FIG. 5 is a front view of the photobioreactor of FIG. 3;

FIG. 6 is a side view of the photobioreactor of FIG. 4;

FIGS. 7A, 7B and 7C are curves showing respectively the cells diameter as a function of time (7A), the cells volume as a function of time (7B), and the pH (7C) as a function of days.

### DETAILED DESCRIPTION OF THE DISCLOSURE

The following examples are presented in a non-limitative manner.

For example, the injector can be an elongated member provided with apertures for injecting a gas inside bag, the member being disposed inside the bag on the first wall. For example, the injector can be integrated into the first wall, molded into the first wall or connected to the first wall. For example, the elongated member can be a tubular member. For example, the bag can comprise a plurality of injectors. The injector(s) can be effective for generating gas bubbles of various sizes. For example, the injector(s) are effective for preventing microalgae from substantially clumping together or forming aggregates. For example, the injector(s) can be effective for generating a dynamic movement or circulation or stream into the culture medium, thereby preventing or at least reducing the agglutination of microalgae of formation of aggregates and provide enough hydrodynamics for biofilm limitation.

For example, the at least one injector can be an elongated member provided with apertures for injecting a gas inside the bag, the member being disposed inside the bag, on the at least one wall, at the second end portion.

For example, the elongated member can be a tubular member.

For example, the at least one injector can be integrated into the at least one wall.

For example, the at least one injector can be molded into the at least one wall or connected thereto.

For example, the first and second end portions can be opposite end portions.

For example, the bag can comprise at least two walls sealingly connected together and defining the internal chamber. The at least two walls can have each portions substantially defining boundaries of the walls and the portions of one wall are sealingly connected with corresponding portions of another wall.

For example, the at least two walls can have a general square or rectangular shape and wherein the bag optionally comprises a single piece or two different pieces.

For example, the first end portion can be a top portion and the second end portion is a bottom portion.

For example, the at least one port can be disposed on the at least one wall in an intermediate portion located between the first and second portions.

For example, the bag can comprise at least three walls that are a top wall, a side wall and a bottom wall and wherein the at least one inlet is disposed on the top wall or adjacently thereto, and the at least one injector is disposed on the bottom wall or adjacently thereto.

For example, the at least one outlet can be disposed on the side wall or adjacently thereto.

For example, the at least one outlet is disposed on the bottom wall or adjacently thereto.

For example, the bag can comprise at least six walls that are a top wall, four side walls and a bottom wall and wherein the at least one inlet is disposed on the top wall or adjacently thereto, and the at least one injector is disposed on the bottom wall or adjacently thereto.

For example, the at least one outlet can be disposed on one of the side walls or adjacently thereto.

For example, the at least one outlet can be disposed on the bottom wall or adjacently thereto.

For example, the bag can have a parallelepiped shape.

For example, the bag can have rectangular prism shape.

For example, the bag can have rounded corners.

For example, the at least one inlet can comprise a valve.

For example, the at least one outlet for harvesting a content of the bag comprises a valve.

For example, the bag can be a disposable bag.

For example, the bag can be sterilized.

For example, the bag can be sealed under sterile conditions.

For example, the bag can be made of a flexible polymer.

For example, the bag can have a thickness of less than 0.6, 0.3 or 0.2 mm.

For example, the bag can have a thickness of about 0.1 to about 0.2 mm.

For example, the bag can comprise polyethylene.

For example, the outlet can be disposed adjacently to a junction of the second wall and the first side wall.

For example, the first and second walls of the bag can be opposite walls. The outlet can be disposed adjacently to the first side wall or adjacently to a second side wall that is opposite to the first side wall.

For example, the outlet can be disposed adjacently to a junction of the second wall and a second side wall that is opposite to the first side wall. For example, the first wall can be a top wall and the second wall can be a bottom wall.

For example, the bag can have rounded corners. The inlet of the first wall can comprise a valve and/or the outlet for harvesting a content of the bag can comprise a valve. For example, the bag can be a disposable bag.

For example the bag is effective for maintaining the culture medium under sterile conditions. For example, the bag can have an internal surface effective for preventing microalgae from sticking thereto or from being agglutinated thereto.

For example, the culture bag used in the photobioreactor can be a bag as defined in the present disclosure.

For example, the at least one lighting element can be a LED lighting element (such as a white LED, a blue LED or a mixture thereof). Alternatively, the lighting element can be an organic light emitting diode (OLED).

For example, the housing can comprise at least one wall provided with a plurality of translucent or transparent portions and a plurality of opaque portions, the translucent or transparent portions and the opaque portions can be disposed in an alternating manner.

For example, the portions can be vertically extending portions disposed in an alternating manner.

For example, the translucent or transparent portion can be a window having 1/100, 1/75, 1/50, 1/20, 1/10, or 1/5 of the total surface area of a wall. Alternatively, the translucent or transparent portion can represent about 80 to about 100% of the total surface of a wall i.e. substantially the whole wall can be translucent or transparent.

For example, the housing can comprise two pairs of opposite walls in which at least one of the walls is provided with the translucent or transparent portions and the opaque portions disposed in an alternating manner. Alternatively, each of the opposite walls can be substantially fully transparent or translucent.

For example, the housing can comprise two pairs of opposite walls in which at least two opposed walls are provided with the translucent or transparent portions and the opaque portions are disposed in an alternating manner.

For example, the housing can comprise a supporting member disposed around the two pairs of opposite walls, the supporting member being disposed in such a manner that the at least one of the walls or the at least two of the walls are disposed between the bag and the supporting member.

For example, the supporting member can be connected to the two pairs of opposite walls.

For example, the housing can comprise at least one wall provided with at least one translucent or transparent portion and the at least one opaque portion is absent, the translucent or transparent portion covering substantially all the surface of the at least one wall.

For example, the housing can comprise at least one wall provided with a plurality of translucent or transparent portions and the at least one opaque portion is absent, the translucent or transparent portions covering substantially all the surface of the at least one wall.

For example, the housing can have a parallelepiped shape

For example, the housing can have a rectangular prism shape.

For example, the photobioreactor can comprise a plurality of lighting elements disposed vis-à-vis the translucent or transparent portions.

For example, the photobioreactor can comprise a plurality of lighting elements that are connected to a bottom wall of the housing and that are disposed vis-à-vis the translucent or transparent portions.

For example, the photobioreactor can comprise a plurality of lighting elements that are connected to a bottom wall of the housing and that are vertically extending and disposed vis-à-vis the translucent or transparent portions.

For example, the translucent or transparent portions can be windows and at least one of the windows can be a pivotable or movable so as to be open.

For example, the photobioreactor can be a vertically extending bioreactor and wherein growing the microalgae can be carried out by injecting a gaseous mixture comprising air and CO₂ at a bottom portion of the photobioreactor and by illuminating the photobioreator with LEDs (such as a white LED, a blue LED or a mixture thereof).

The lighting used can be, for example, white and blue electroluminescent diodes (LEDs) that adapt to standard receptacles for T-8 fluorescent tubes and emit an intensity of approximately 8,000 to 10,000 lux with a wavelength of 400 to 700 nm. The tubes can also have an intensity of about 5000 to about 9000 K. The lighting element can be provided with wavelength that can be specific to photo-pigments present in produced species. The LED tubes can be mounted on the housing in notches or spaces adapted therefore.

According to another aspect, there is provided a photobioreator modular system comprising a plurality of photobioreactor units wherein at least two of the units are a photobioreactor as defined in the present disclosure and wherein the at least two units are connected together by means of connecting elements.

For example, the connected photobioreactors can be slot in a spatial structure with structural functions made of two levels; one on the floor for footing, and one at top, as a mezzanine with footbridges that allow access to photobioreactor and that can support walls of photobioreactor full of culture medium. The connecting elements between two photobioreactors can be made of a material that can resist to corrosion such as fibreglass.

For example more than one photobioreactors can share a common culture bag.

For example, the at least one photobioreactor can have at least one removable wall that is optionally removed when combining it with another photobioreactor so as to put their respective internal chamber in fluid flow communication with one another.

For example, the photobioreactors of a same units can be connected together in such a manner that a user has access to the internal chamber of each of the photobioreactors.

For example, the bags of the photobioreactors can be dimensioned in such a manner that each unit comprises a single bag or a plurality of bags.

For example, a top portion of the unit can be provided with a mezzanine-type structure that facilitates access to the various internal chambers and facilitating connecting the photobioreactors with one another.

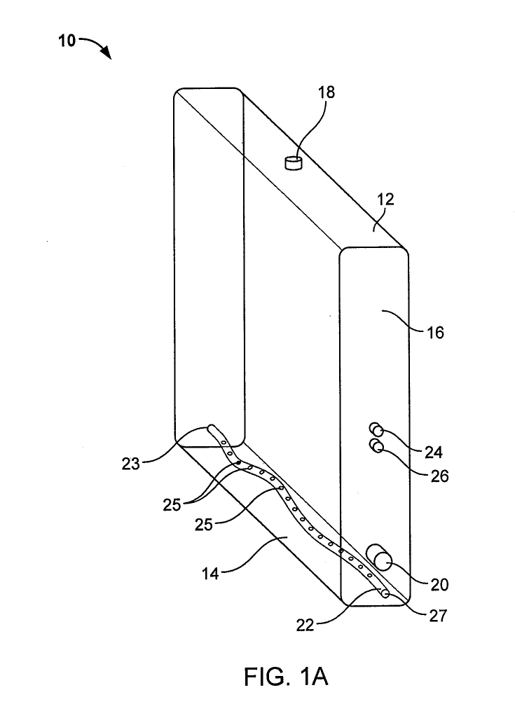

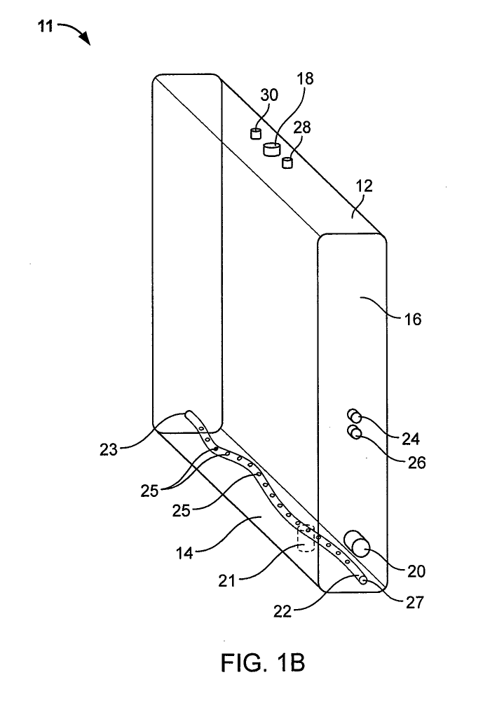

As it can be seen in FIG. 1A, the culture bag 10 can be provided with a first wall (for example top wall 12), a second wall (for example bottom wall 14), and side walls 16. The top wall 12 can comprise an inlet 18 for filling the bag with a culture medium. The inlet 18 can also act as a gas outlet for exhausting and/or recovering gases. The inlet 18 can be provided with a valve. Further inlets (or outlets) can also be provided For example, the inlet 18 can be provided with a valve. One of the side walls 16 (or the bottom wall 14) can comprise an outlet 20 for harvesting the microalgae. For example, the outlet 20 can be provided with a valve. The bag 10 can be provided with an injector 22 that can be disposed on the bottom wall 14, connected thereto, integrated therein, or molded thereto. The injector can be a tube provided with apertures 25 for injecting a gas. The injector 22 can comprise a cap 27 for closing an end portion. The apertures can be provided at every 5, 10 or 15 cm. The apertures can be more numerous at the extremities for generating an example of a gas distribution pattern inside the bag. The various walls can comprise of minimum sheet(s) (or layers) of plastic or polymer material. The walls can also be sealed to insure sterile conditions.

The bag 10 can optionally be provided with inlets (also called apertures or ports) 24 and 26. The ports 24 and 26 can be provided with valves and can be useful for inserting a sensor, a probe and/or a sampling loop. The inlet 18 can also be suitable to insert sensor proposed to be inserted at port 24 and 26. The bag can have rounded corners and all the walls can be sealingly connected together. The bag can comprise polypropylene. The bag can also be made of various polymers or materials that are translucent or transparent so as to allow passage of light. For example, passage of light can be allowed without substantially modifying the spectrum of light.

The bag 11 illustrated in FIG. 1B is similar to the bag 10 of FIG. 1A. Several reference numbers are the same since representing the same or similar components. However in FIG. 1B, a further outlet 21 is provided. The outlet 21 as the same function than outlet 20 previously discussed. The bag 11 can also optionally be provided with further ports 28 and 30 provided on the top wall 12. The ports 28 and 30 have the same functions than ports 24 and 26 (they are equivalents).

When using the culture bag 10 or 11, the bag can be provided as a sterilized bag. The bag can be filled with the culture medium via the inlet 18 and then, the microalgae can be grown. When completed, the microalgae can be harvested via the outlet 20. The bag can then be washed before being recycled or be disposed.

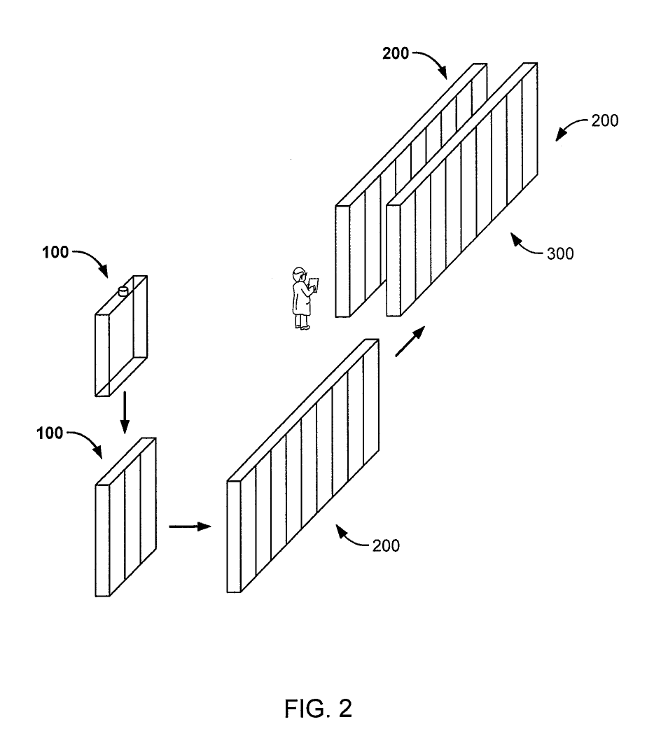

As it can be seen from the schematic representation of FIG. 2, the culture bag 10 or 11 is inserted in the photobioreactor 100. Three photobioreactors 100 (or three units) can be connected together to form a set 200 (or a row 200). Two sets of photobioreactors 200 (or two rows of photobioreactors 200) can be combined together to obtain a photobioreactor modular system 300. The photobioreactor modular system 300 in fact can comprise a plurality of sets disposed in various manner (thus implicitely a plurality of photobioreactors).

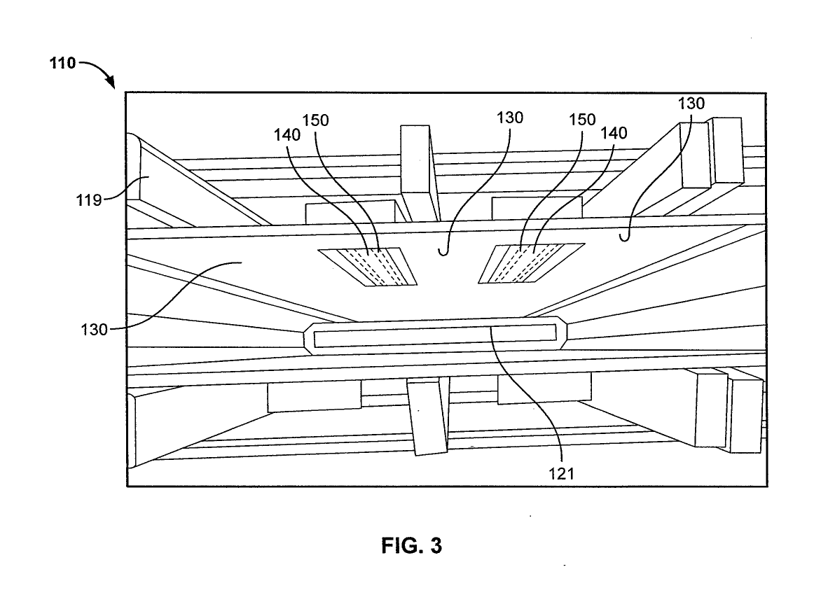

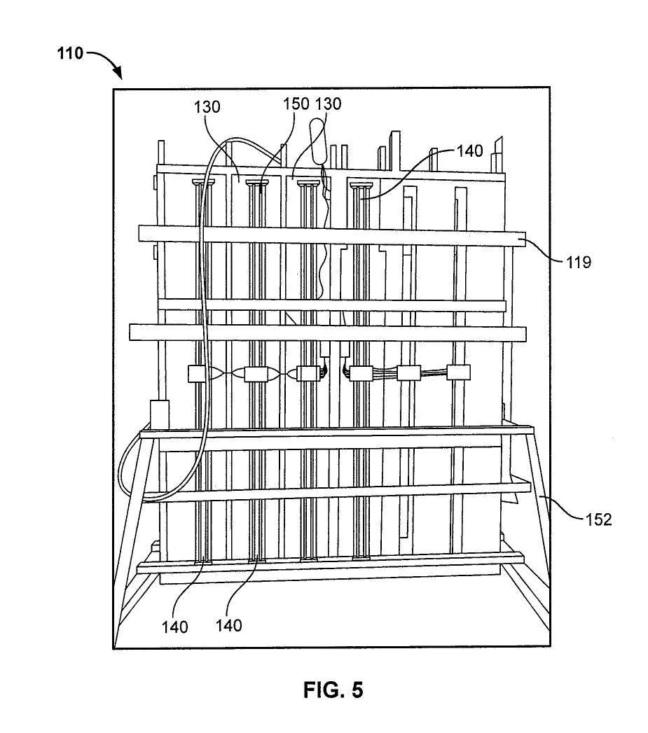

As it can be seen from FIG. 3, a housing 119 of a photobioreactor 110 that defines an internal chamber (121) dimensioned for receiving a culture bag (not shown). The protobioreactor 110 can comprise opaque portions 130 and translucent or transparent potions 140 that are disposed in an alternating manner (i.e. translucent portion 140—opaque portion 130—translucent portion 140—opaque portion 130 and so on . . . ). These portions can all be vertically extending. The lighting elements 150 (for example LED lighting elements) can be disposed vis-à-vis the translucent or transparent potions. The uppermost wall with respect to the position of the photobioreactor in the picture of FIG. 3 clearly show the alternating portions 130 and 140, the lighting elements 150 being disposed vis-à-vis the transparent or translucent portions 140. This pattern can also be seen from FIG. 5 in which the lighting elements 150 are seen from behind (if the portion of lighting elements as seen in FIG. 3 is considered as the front portion of these lighting elements). The alternating portions 130 and 140 of the photobioreactor 110 are also clearly seen from FIG. 5. The opaque portions can be made of various materials that are opaque and suitable for acting as walls defining the internal chamber. The transparent or translucent portions can be made of various materials effective for allowing passage of light from the lighting elements 150 to the internal chamber 121 defined by the photobioreactor 110. The photobioreactor 110 also comprises a support member 152.

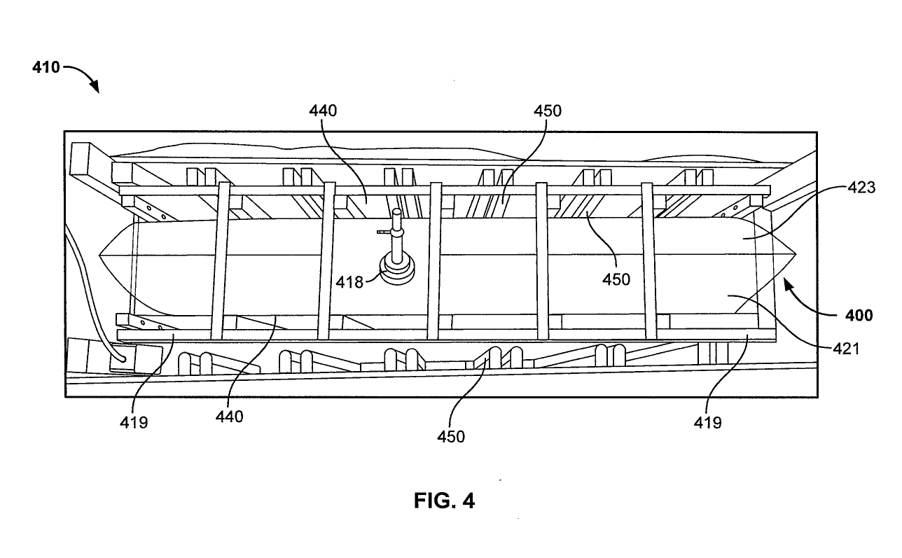

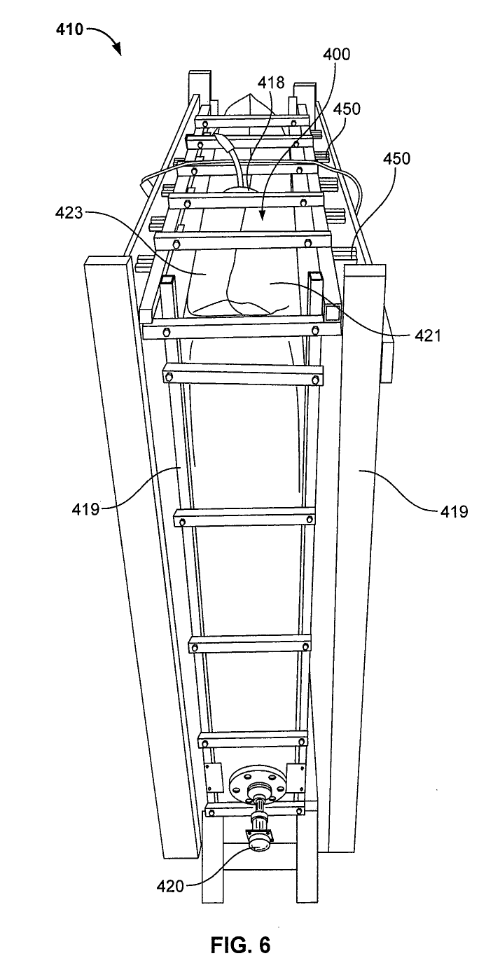

In FIGS. 4 and 6, a culture bag 400 has been inserted in a photobioreactor 410. The photobioreactor 410 comprises a housing 419 including transparent or translucent portions 440 (for example, fiberglass can be used). The photobioreactor 410 also comprises lighting elements 450 (for example LED lighting elements) The photobioreactor 410 does not comprise opaque portions. The culture bag 400 is provided with an outlet valve 420. In the case of the photobioreactor 410, a cover can be further provided to cover the entirety of the housing 419 (not shown) so as to prevent considerable losses of light. This cover can be provided with a material that can reflect light. As it can be seen from FIGS. 4 and 6, the bag 400 comprises only two walls 421 and 423 that are sealingly connected together and they define the internal chamber. The walls 421 and 423 have each portions that substantially define the boundaries of these walls and for example, the boundary portions of wall 421 are sealingly connected with the corresponding portions of wall 423.

For example, the microalgae can be phototrophic microalgae. For example, the microalgae can be autotrophic microalgae. For example, the microalgae can be mixotrophic microalgae. For example, the microalgae can be marine microalgae or fresh water microalgae. The microalgae can be chosen from Isochrysis galbana, Pavlova lutheri, Nannochloropsis oculata, Chaetoceros muelleri, Skeletonema costatum, Rhodomonas Tetraselmis suesica, Phaeodactylum tricornutum, Chlorella vulgaris, Spirulina platensis, and Thalassiosira weissflogii. For example, the microalgae can be Pavlova lutheri. For example, the microalgae can be Nannochloropsis oculata.

For example, the culture medium can be prepared by filtering and/or sterilizing seawater and mixing the filtered seawater with nutrients effective for feeding microalgae thereto.

For example, the microalgae can have been inoculated into the photobioreactor before introducing the culture medium therein. For example, the microalgae can have been inoculated, in sterile condition from axenic inoculum, into the photobioreactor before introducing the culture medium therein. The culture medium can be inserted only once, continuously or semi-continuously depending on the production mode. Of course, some portions of the content of the bag will be removed (harvested) to compensate further additions of inoculum.

### EXPERIMENTAL DATA

### Example 1

The photobioreactor as shown in FIGS. 3 and 5 was used for the following experiments. The supporting member was built in wood but any other material suitable for supporting the walls defining the internal chamber adapted to receive the bag can be used. For example, fiberglass can be used. The example used measures 9″ high, 9″ wide and contains an internal space of 8″ in width. The inside corners are lined with foam blocks, in a tapered form, so as to avoid right angles. It was flanked by four (4) LED tubes, spaced apart every 12″. The LED tubes come from the company LESS, measure four (4) feet in length, and emit an light intensity of 40 000 lux/tub (T8L4-18-FL-85˜265Vac-5500K). The translucent or transparent portions were windows made of Plexiglas that causes a reduction in the order of 25% of light. The plastic bag comprising polypropylene was inserted into the interior of the photobioreator (internal chamber) through its top. The bag provoked a decrease in light in the order of 20%. The amount of light transmitted to the culture by the LED tube was therefore 26 000 lux. A Supply-Harvest-Bubbling-Sampling (SHBS) system was especially designed to operate the bag production; the latter did not contain any valves. It was inserted through the top of the bag.

A new bag was thus inserted into the internal chamber of the photobioreactor through the top opening. It is worth noting that for the present test, only bags measuring 6″ in length were available, forcing the applicants to reduce the internal chamber to 8″×6″×9″ (for a useful or working volume of 800 L). The SHBS system was then placed on the bottom of the bag. The bag was filled with javel water 200 ppm (via SHBS), closed with clips, which tightly clamped the surplus rolled portion of the bag, and left for 24 hours of sterilization. The air outlet was situated around the corresponding inlet of the SHBS system. When using the culture bag as shown in FIG. 1A, such a situation is different since the bag of FIG. 1A is sealingly closed (no need of clamps). Moreover, the bag of FIG. 1A can comprise valves.

Once the sterilization was conducted, the bag was rinsed twice with the new culture medium before being sown with 50 liters of Nannochloropsis oculata culture. The culture used to inoculate the photobioreactor was aged of 8 days. The cellular growth was followed daily by conducting cellular counts with a particle counter (Z2 Beckman, volume and cellular concentration). The pH was measured daily with a pH meter. The culture was produced by carrying out three (3) harvests per week with a dilution factor of 20 or 10×10⁶ cell/ml. The cultures were placed under light 24 hr/24 hr, but the light intensity was reduced to 50% following 6 hours after the harvest in order to avoid photo-inhibition phenomena following density modification.

The experiment was conducted for 10 days. During the experiment, the average cellular volume was about 14±1 μm³ and the average pH was about 7.9±0.5. It was possible to harvest 20×10¹² cells (±3×10¹²) for an average volume of 500 liters. This experiment allowed for validating the concept of autotrophic production of microalgae in an example of a photobioreactor as described in the present disclosure. The characteristics of the culture (cellular volume and pH) were stable for the duration of the experiment and are comparable to those obtained in a cylindrical photobioreactor such as one as described in PCT/CA2011/001216, which in hereby incorporated by reference in its entirety. However, to produce a given quantity of microalgae, the photobioreactor of the present disclosure required a smaller footprint.

### Example 2

Another example similar to example 1 was carried out with a photobioreactor as shown in FIGS. 4 and 6. The bag used was made with a polyethylene film having a thickness of about 0.15 mm sold under the tradename of Ultra Plus Vapour Barrier™ by Duchesne, Yamachiche, Quebec, Canada. The film was sealed by using a Seal a Meal™ sealing machine. The culture bag has a working volume of about 300 L. The LED lighting elements were disposed at a distance of about 7.5 cm from bag. The combination of the translucent or transparent portions (made of Plexiglas) and the polyethylene bag caused a reduction in the order of 20% of light.

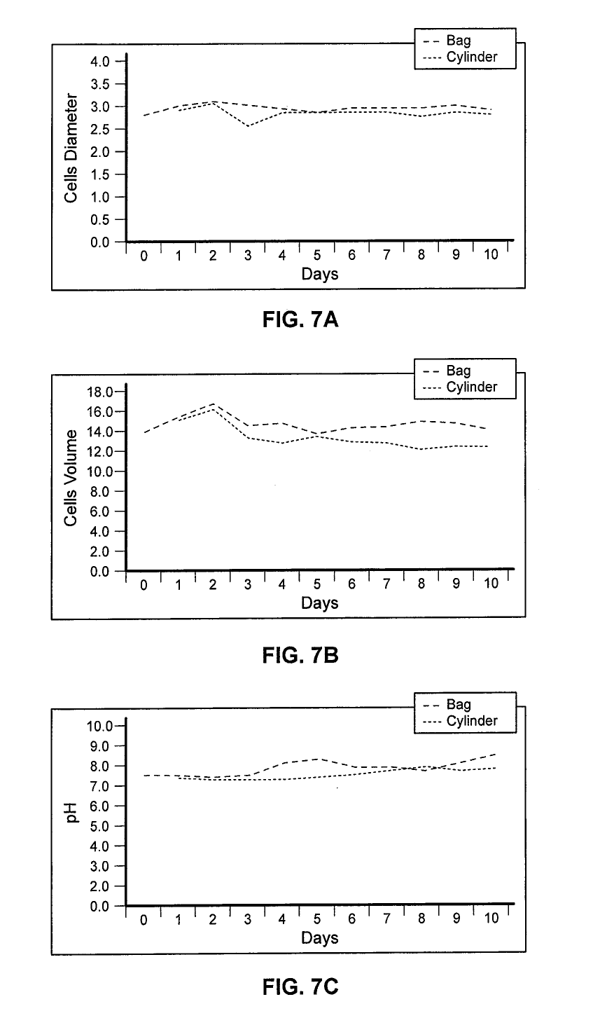

In example 2, a comparison of the photobioreator of FIGS. 4 and 6 (see “bag” on FIGS. 7A, 7B and 7C) was made with the photobioreactor described in PCT/CA2011/001216 (cylindrical photobioreactor (see “cylinder” of FIGS. 7A, 7B and 7C) and having a volume of 340 L. The photobioreator of FIGS. 4 and 6 was inoculated with 20 L of liters of Nannochloropsis oculata culture and the cylindrical photobioreactor under similar conditions.

The footprint of the photobioreactor of FIGS. 4 and 6 was about 0.8 m² and the footprint of the cylindrical photobioreactor was about 1 m².

After 8 days in the bag, the culture medium did not show any aggregates of microalgae, protozoa or visible bacteria.

As it can be seen from FIGS. 7A, 7B and 7C, the results obtained with the bag photobioreactor of FIGS. 4 and 6 are similar to the results obtained with the cylindrical photobioreactor of PCT/CA2011/001216. The cells diameter was (see FIG. 7A) and the cells volume (see FIG. 7B) were greater for the cells produced with the bag photobioreactor and the pH (see FIG. 7C) was about the same in both cases. It was thus demonstrated that the bag photobioreactor can be at least as much efficient than the cylindrical photobioreactor of PCT/CA2011/001216 and even superior. It was thus demonstrated that the photobioreactors and the bags described in the present disclosure represent an efficient alternative for producing microalgae. In fact, such bags can be produced at low costs, such photobioreactors allow for reducing the footprint while offering a high efficiency for producing microalgae.

It was found that the bags and photobioreactors of the present disclosure are effective for optimizing the volume of culture produced per unit of area (for example square foot of a floor of building required or occupied by the photobioreactor). In other words, the bags and photobioreactors of the present disclosure only require a small footprint. Moreover, it was found that these bags and photobioreactors allowed for better homogenization of the culture, which renders its control easier to handle by an automate. In order to reduce the cleaning time of the photobioreactors, it is possible to use recyclable bags to contain the culture. It can thus be the that such bags and photobioreactors are quite efficient.

Moreover, it was found that the bags and photobioreactors were quite effective for minimizing the lost of light. In fact, it was observed that the opaque portions were quite effective for retaining light by reflecting light inside the photobioreactors. For example, it was observed that the fact of having the opaque portions and the translucent or transparent portions disposed in an alternating manner allowed for considerably lowering the loss of light.

The bags and photobioreactors were also found to be effective for providing a high level of cell concentration, an easy operation while allowing for a continuous production.

The fact that such bags do not necessitate a cleaning step allows for saving a considerable amount of time which generates an increased production capacity. Another factor increasing the production capacity is the fact that the photobioreactors of the present disclosure have a very high volume production capacity per each square meter that it occupied on a floor of a building. In fact, such a technology allows for a given quantity of microalgae produced, to reduce the footprint occupied by the system used for producing the microalgae. In other words, this technology allows for increasing the amount of microalgae produced for each square meter (footprint) occupied by the production system.

The photobioreactors thus provide the sturdiness while integrating the lightning system in the structure and keeping a maximum amount of photons totally in the culture. This allows a maximization use of the light used to operate the photobioreactors. The cleaning of a conventional photobioreactor is always an important cost component of the operation and restrains the development of a cost effective supply of microalgae and for example of its vegetable omega 3 source. The bags of the present disclosure thus allow to overcome such a drawback. These bags increase drastically the production capacity and reduce the closure and start up efforts since there is no more need to clean the photobioreactors.

The person skilled in the art would understand that the various properties or features presented in a given embodiment can be added and/or used, when applicable, to any other embodiment covered by the general scope of the present disclosure.

The present disclosure has been described with regard to specific examples. The description was intended to help the understanding of the disclosure, rather than to limit its scope. It will be apparent to one skilled in the art that various modifications can be made to the disclosure without departing from the scope of the disclosure as described herein, and such modifications are intended to be covered by the present document.

1. A culture bag for use in a photobioreactor, said bag comprising:
at least one wall having at least one inlet disposed at a first end portion of said at least one wall or adjacently thereto, said at least one wall defining an internal chamber for receiving a culture medium;
at least one injector for injecting a gas inside said bag, said at least one injector being disposed at a second end portion of said at least one wall or adjacently thereto;
optionally at least one port disposed on said at least one wall of said bag, said at least one port being effective for receiving at least one element chosen from a sensor, a sampling loop and a probe; and
at least one outlet for harvesting a content of said bag, said at least one outlet being disposed at said second end portion of said at least one wall or adjacently thereto,
said bag being translucent or transparent and being effective for holding and sealingly maintaining said culture medium inside said bag and inside said photobioreactor.

2. The bag of claim 1, wherein said at least one injector is an elongated member provided with apertures for injecting a gas inside said bag, said member being disposed inside said bag, on said at least one wall, at said second end portion.

3. (canceled)

4. The bag of claim 1, wherein said at least one injector is integrated into said at least one wall.

5. The bag of claim 1, wherein said at least one injector is molded into said at least one wall or connected thereto.

6. (canceled)

7. The bag of claim 1, wherein said bag comprises at least two walls sealingly connected together and defining said internal chamber, said at least two walls having each portions substantially defining boundaries of said walls and said portions of one wall are sealingly connected with corresponding portions of another wall.

8. The bag of claim 7, wherein said at least two walls have a general square or rectangular shape and wherein said bag optionally comprises a single piece or two different pieces.

9. The bag of claim 8, wherein said first end portion is a top portion and said second end portion is a bottom portion.

10. The bag of claim 9, wherein the at least one port is disposed on said at least one wall in an intermediate portion located between said first and second portions.

11. The bag of claim 7, wherein said bag comprises at least three walls that are a top wall, a side wall and a bottom wall and wherein said at least one inlet is disposed on said top wall or adjacently thereto, and said at least one injector is disposed on said bottom wall or adjacently thereto.

12-13. (canceled)

14. The bag of claim 7, wherein said bag comprises at least six walls that are a top wall, four side walls and a bottom wall and wherein said at least one inlet is disposed on said top wall or adjacently thereto, and said at least one injector is disposed on said bottom wall or adjacently thereto.

15-23. (canceled)

24. The bag of claim 14, wherein said bag is sealed under sterile conditions.

25-29. (canceled)

30. The bag of claim 24, wherein said bag comprises polyethylene.

31. A photobioreactor comprising:
a culture bag dimensioned for receiving a culture medium;
a housing defining an internal chamber dimensioned for receiving said culture bag, said housing comprising at least one wall having at least one translucent or transparent portion comprising a translucent or transparent material and optionally at least one opaque portion comprising at least one opaque material; and
at least one lighting element disposed adjacently to said translucent or transparent portion so as to provide light inside said chamber.

32. (canceled)

33. The photobioreactor of claim 32, wherein said at least one lighting element is a LED lighting element.

34. The photobioreactor of claim 33, wherein said housing comprises at least one wall provided with a plurality of translucent or transparent portions and a plurality of opaque portions, said translucent or transparent portions and said opaque portions are disposed in an alternating manner.

35. The photobioreactor of claim 34, wherein said portions are vertically extending portions disposed in an alternating manner.

36. The photobioreactor of claim 35, wherein said housing comprises two pairs of opposite walls in which at least one of said walls is provided with said translucent or transparent portions and said opaque portions disposed in an alternating manner.

37. (canceled)

38. The photobioreactor of claim 36, wherein said housing comprises a supporting member disposed around said two pairs of opposite walls, said supporting member being disposed in such a manner that said at least one of said walls or said at least two of said walls are disposed between said bag and said supporting member.

39. (canceled)

40. The photobioreactor of claim 33, wherein said housing comprises at least one wall provided with at least one translucent or transparent portion and said at least one opaque portion is absent, said translucent or transparent portion covering substantially all the surface of said at least one wall.

41. The photobioreactor of claim 33, wherein said housing comprises at least one wall provided with a plurality of translucent or transparent portions and said at least one opaque portion is absent, said translucent or transparent portions covering substantially all the surface of said at least one wall.

42-54. (canceled)
